(19) United States
(12) Patent Application Publication
(10) Pub. No.: US 2024/0325592 A1
(43) Pub. Date: Oct. 3, 2024

(54) LIQUID EMBOLIC MATERIALS

(71) Applicant: Boston Scientific Scimed, Inc., Maple Grove, MN (US)

(72) Inventors: Nicolas Ball-Jones, Minneapolis, MN (US); Steven L. Kangas, Woodbury, MN (US); Samuel Michael Hanson, Minneapolis, MN (US)

(73) Assignee: Boston Scientific Scimed, Inc., Maple Grove, MN (US)

(21) Appl. No.: 18/618,015

(22) Filed: Mar. 27, 2024

Related U.S. Application Data
(60) Provisional application No. 63/492,377, filed on Mar. 27, 2023.

(51) Int. Cl. A61L 24/04

(52) U.S. Cl. CPC A61L 24/046; A61L 2400/04; A61L 2400/06

(57) ABSTRACT

Aqueous liquid embolic compositions, for example formed from amino acids, as well as method for making and using aqueous liquid embolic compositions are disclosed. An example aqueous liquid embolic composition may include a di-block or tri-block copolymer including a charged block and a hydrophobic block. The aqueous liquid embolic composition may also include templating agent.

### CROSS-REFERENCE TO RELATED APPLICATIONS

This application claims the benefit of U.S. Provisional Patent Application Ser. No. 63/492,377 filed on Mar. 27, 2023, the disclosure of which is incorporated herein by reference.

### TECHNICAL FIELD

The present disclosure pertains to liquid embolic materials.

### BACKGROUND

A number of embolic materials have been developed for medical use, for example, intravascular use. Of the known embolic materials, each has certain advantages and disadvantages. There is an ongoing need to provide alternative embolic materials.

### BRIEF SUMMARY

This disclosure provides design, material, manufacturing method, and use alternatives for aqueous liquid embolic materials. An aqueous liquid embolic material is disclosed. The aqueous liquid embolic material comprises: a di-block or tri-block copolymer including a charged block and a hydrophobic block; and a templating agent.

Alternatively or additionally to any of the embodiments above, wherein the charged block includes polylysine.

Alternatively or additionally to any of the embodiments above, the charged block includes polyornithine.

Alternatively or additionally to any of the embodiments above, the charged block includes polyarginine.

Alternatively or additionally to any of the embodiments above, the charged block includes polymethylarginine.

Alternatively or additionally to any of the embodiments above, the charged block includes polyglutamic acid.

Alternatively or additionally to any of the embodiments above, the charged block includes polyaspartic acid.

Alternatively or additionally to any of the embodiments above, the charged block includes a natural or non-natural amino acid with a pKa from 3-11.

Alternatively or additionally to any of the embodiments above, the hydrophobic block includes a hydrophobic amino acid.

Alternatively or additionally to any of the embodiments above, the hydrophobic block includes polyphenylalanine.

Alternatively or additionally to any of the embodiments above, the hydrophobic block includes one or more of polyleucine, polyisoleucine, polytryptophan, and polytyrosine.

Alternatively or additionally to any of the embodiments above, the hydrophobic block includes one or more natural or non-natural amino acids that have pendant aliphatic, cycloaliphatic, or aromatic side chains.

Alternatively or additionally to any of the embodiments above, the composition becomes a gel when injected into a vasculature of a patient.

Alternatively or additionally to any of the embodiments above, the composition includes a di-block copolymer.

Alternatively or additionally to any of the embodiments above, the composition includes a tri-block copolymer.

Alternatively or additionally to any of the embodiments above, the templating agent includes one or more of gelatin, hyaluronic acid, carboxy methylcellulose, poly(2-acrylamido-2-methyl-1-propanesulfonic acid), chitosan, alginate, chondroitan sulfate, polydiallyldimethylammonium chloride, polylysine, polyarginine, polyallylamine, polysodium acrylate, sulfonated polystyrene, polyvinyl sulfonic acid, poly N,N-dimethyl-3,5-dimethylene piperidinium chloride, poly(vinylbenzyl trimethylammonium) chloride, polyvinyl amine, carrageenan, pectin, xanthan gum, and guar gum.

An aqueous liquid embolic composition is disclosed. The aqueous liquid embolic material comprises: a block copolymer including a charged block and a hydrophobic block; wherein the charged block includes a charged amino acid; and wherein the hydrophobic block includes a hydrophobic amino acid.

Alternatively or additionally to any of the embodiments above, the block copolymer includes a di-block copolymer.

A kit for embolization is disclosed. The kit comprises: a syringe containing a block copolymer including a charged block and a hydrophobic block; wherein the charged block includes a charged (cationic or anionic) amino acid; wherein the hydrophobic block includes a hydrophobic amino acid; and a templating agent.

Alternatively or additionally to any of the embodiments above, the templating agent is disposed within the syringe.

Alternatively or additionally to any of the embodiments above, further comprising a second syringe containing the templating agent.

The above summary of some embodiments is not intended to describe each disclosed embodiment or every implementation of the present disclosure. The Detailed Description, which follows, more particularly exemplify these embodiments.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant FIGURE.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

Liquid embolic systems have gained increasing acceptance as effective agents for the embolization or filling of neural and peripheral spaces, such as hyper-vascular tumors, arteriovenous malformations, aneurisms, and endoleaks. Some liquid embolic systems may use solvents such as DMSO. Such systems may have limitations due to the intrinsic toxicity of DMSO which can cause tissue necrosis and vessel spasm. Because of this, it may be desirable to utilize an aqueous based system. In addition to being able to avoid using DMSO, aqueous system may have greater biocompatibility, case of use, and the potential for drug loading applications. Disclosed herein are compositions and systems such as liquid embolic compositions/materials and/or systems. Such liquid embolic compositions are generally suitable for injection into the body and may be provided in a vial or syringe barrel. In some instances, the material/composition in the vial or syringe barrel may be a liquid material/composition, for example, an aqueous embolic liquid material/composition. Some additional details of example aqueous embolic liquid materials/compositions are disclosed herein.

In some instances, example embolic compositions may include liquid compositions that form a gel material (also referred to herein as a hydrogel material or a solidified material) in situ upon injection into the body. Such liquid compositions include liquid compositions that are capable of gel formation in response to in vivo conditions. In some instances, the liquid compositions may form gels in response to a change in pH and/or a change in temperature. The compositions may be in liquid form at room temperature (e.g., at or below 25° C., or at or below 30° C. in some instances), and may have a pH that is less than or equal to about 7.0, typically ranging from about 4.5 to 6.5, or from about 5 to 6. After delivery to the body of a patient (e.g., to in vivo conditions where the temperature is about 37° C. and the pH is about 7) the liquid compositions spontaneously form a gel. In other instances, a shear thinning mechanism can be harnessed to create the embolic material in vivo.

Such liquid compositions can be used in a number of medical applications, including use as liquid embolic compositions, fiducial markers, tissue-bulking materials, tissue-spacing materials, and depots which comprise a therapeutic agent and from which the therapeutic agent elutes into the surrounding tissue. In instances where the liquid compositions are injected into the body of a subject, the liquid compositions may be adapted to pass through the particular delivery device employed for the injection, preferably, with manual pressure. For example, in a typical injection, with the thumb pushing on the plunger and the ipsilateral index and middle fingers stabilizing flanks of the syringe barrel, an injection force of less than 50 N is preferred. The desired viscosity level will typically be dependent on the procedure and the delivery method. For direct injection with a needle and syringe, the amount of pressure required will depend, for example, on the gauge of the needle. Similarly, for injection via catheter, the amount of pressure required will depend, for example, upon the catheter internal diameter.

In some stances, the liquid compositions of the present disclosure have a viscosity ranging from about 10 mPa·s or less to about 5000 mPa·s or more when measured at shear rate 50 1/s at a temperature of 25° C. For example the compositions may have a viscosity ranging anywhere from about 10 mPa·s to 25 mPa·s to 50 mPa·s to 100 mPa·s to 250 mPa·s to 500 mPa·s to 1000 mPa·s to 2500 mPa·s to 5000 mPa·s at a shear rate 50 1/s and a temperature 25° C. (e.g., ranging between any two of the preceding numerical values).

The aqueous liquid embolic materials/compositions disclosed herein may include block copolymer. For example, the block copolymer may be a di- or tri-block copolymer. In other instances they can include additional blocks (e.g. a tetra-block and beyond). The block copolymer may be represented by the general formula A-B (e.g., for a di-block copolymer) or A-B-A (e.g., for a tri-block copolymer) where A represents a charged block and B represents a charged block. These are examples. Other arrangements are contemplated.

In some instances, the charged block may include:

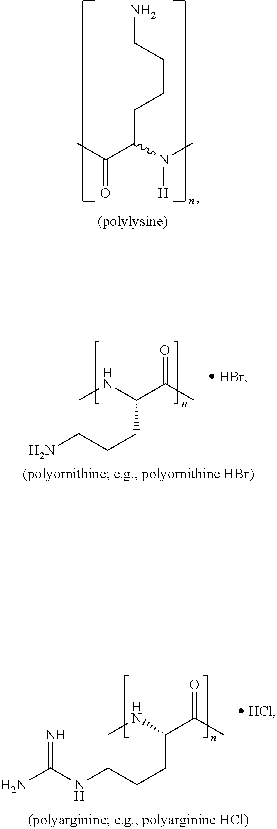

polymethylarginine, polyglutamic acid, polyaspartic acid (e.g., naturally occurring fragments including α-linked L-aspartactic acid and/or synthetic polyaspartic acid in any of four isometric forms), combinations thereof, and/or the like. In some instances, the charged block monomer may have a pKa in the range of about 2-11, or about 3-10. This may help to provide the aqueous liquid embolic composition with a desirable pH (e.g., in the range of about 4-11.5, or about 5-11, or about 5-10). Additional natural and non-natural amino acids (having pendant aliphatic, cycloaliphatic, and aromatic functional groups that are charged at pH=3-10) are envisioned for the charged polypeptide block.

In some instances, each of the charged blocks range in length from 5 monomer units or less to 1000 monomer units or more, for example, ranging anywhere from 5 to 10 to 25 to 50 to 100 to 300 to 600 to 1000 monomer units (e.g., ranging between any two of the preceding numerical values). For example, the charged block may be greater than or equal to 100 monomer units in length and less than or equal to 300 monomer units in length.

It can be appreciated that in some instances, the charged blocks may include a charge. For example, the charged blocks may include one or more cations under biological conditions. Alternatively, the charged block may include one or more anions under biological conditions. The charge of the charged blocks may allow the block copolymer to be loaded with charged drugs. For example, cationic charged blocks may be loadable with negatively charged drugs. Anionic charged blocks may be loadable with positively charged drugs.

In some instances, the hydrophobic block may include one or more amino acids, a hydrophobic amino acid (e.g., a hydrophobic poly amino acid and/or a hydrophobic polypeptide), polyphenylalanine, iodinated polyphenylalanine, polyleucine, iodinated polyleucine, polyisoleucine, iodinated polyisoleucine, polytryptophan, iodinated polytryptophan, polytyrosine, iodinated polytyrosine, a polypeptide block (e.g., having a pattern that includes arginine-glycine-aspartic acid), blocks including an aromatic group containing one or more amino acids, combinations thereof, and/or the like. Additional natural and non-natural amino acids (having pendant aliphatic, cycloaliphatic, and aromatic functional groups that are non-charged at pH=3-10) are envisioned for the non-charged/neutral polypeptide block.

In some instances, each of the hydrophobic blocks range in length from 5 monomer units or less to 500 monomer units or more, for example, ranging anywhere from 5 to 25 to 50 to 75 to 100 to 200 to 250 to 300 to 500 monomer units in length (e.g., ranging between any two of the preceding numerical values). For example, the hydrophobic block may be greater than or equal to 5 monomer units in length and less than or equal to 150 monomer units in length.

The use of a hydrophobic block may be desirable for a number of reasons. For example, the hydrophobic block(s) may be substantially non-thermoresponsive. In some of these and in other instances, the hydrophobic block(s) may form a substantially permanent micelle that, for example, can be loaded with drugs (e.g., neutral drugs). The use of a hydrophobic block may also improve the manufacturability of the aqueous liquid embolic composition. For example, removing temperature sensitivity may help improve manufacturability.

In instances where the hydrophobic and charged blocks include an amino-acid based block, it can be appreciated that the block copolymer may be substantially biocompatible (e.g., having a relatively high level of biocompatibility).

In instances where the hydrophobic block and charged block contain amino acids they may be synthesized by ring-opening polymerization of N-carboxyanhydrides (NCA polymerization). In some instances, the process may utilize carbamate protection of amines. Polymerization may use an initiator such as ditrimethyl thio amine, a crown ether, and/or with cooling to 0 C.

In some instances, the block copolymer may be or resemble plastic antibodies that may include a combination of polypeptides capable of binding a specific protein.

In some embodiments, a number average molecular weight of the block copolymer may range from about 2000 Da or less to about 500,000 Da or more. For example, a number average molecular weight of the block copolymer may range anywhere from about 2000 Da to about 5000 Da to about 10,000 Da to about 20,000 Da to about 50,000 Da to about 100,000 Da to about 200,000 Da to about 500,000 Da in number average molecular weight (e.g., ranging between any two of the preceding numerical values).

In some embodiments, the block copolymer may be present in the composition at a concentration ranging from 0.3% wt/wt or less to 50% wt/wt or more with respect to the weight of the composition, which may be, for instance, a liquid composition such as an aqueous liquid composition. For example, the copolymer may be present in the composition in a concentration ranging anywhere from 0.3% wt/wt to 2% wt/wt to 5% wt/wt to 10% wt/wt to 20% wt/wt to 30% wt/wt to 40% wt/wt to 50% wt/wt (e.g., ranging between any two of the preceding numerical values).

The block copolymer may be manufactured using a suitable methodology. For example, the block copolymer may be manufactured as a gradient copolymer that is continuously fed into a batch reactor. This may include NCA (N-carboxyanhydride) polymerization of cyclic amino acid derivatives. In some of these and in other instances, polymer tethering/click reactions (and/or reactions that stitch homopolymer together) may be used. This includes tethering reactions that use azide alkyne cycloadditions, polyurethane formations, and the like.

The aqueous liquid embolic materials/compositions may also include a templating agent. Some examples of suitable templating agents may include gelatin, hyaluronic acid, carboxy methylcellulose, poly(2-acrylamido-2-methyl-1-propanesulfonic acid), chitosan, alginate, chondroitan sulfate, polydiallyldimethylammonium chloride, polylysine, polyarginine, polyallylamine, polysodium acrylate, sulfonated polystyrene, polyvinyl sulfonic acid, poly N,N-dimethyl-3,5-dimethylene piperidinium chloride, poly(vinylbenzyl trimethylammonium) chloride, polyvinyl amine, carrageenan, pectin, xanthan gum, guar gum, combinations thereof, and/or the like. In some instances, the templating agent may have a number average molecular weight ranging from 1,000 Da to 10,000,000 Da, for example ranging anywhere from 1,000 Da to 2,000 Da to 5,000 Da to 10,000 Da to 20,000 Da to 50,000 Da to 100,000 Da to 200,000 Da to 500,000 Da to 1,000,000 Da to 2,000,000 Da to 10,000,000 Da (e.g., ranging between any two of the preceding numerical values).

In some instances, the templating agent may include a polyanionic agent that provides a plurality of negatively charged sites to the block copolymer. This may allow the block copolymer to be loaded with positively charged drugs. In such instances, the templating agent may be termed or understood to be an anionic complementary polymer. For example, in instances where the charged block includes one or more cations, the templating agent may include complementary anions (e.g., the templating agent may be an anionic complementary polymer or material). Alternatively, the templating agent may include a polycationic agent that provide a plurality of positively charged sites to the block copolymer. This may allow the block copolymer to be loaded with negatively charged drugs. In such instances, the templating agent may be termed or understood to be a cationic complementary polymer.

In some instances, the templating agent may be present in an amount ranging from 0.1% wt/wt or less to 50% wt/wt or more with respect to the weight of the composition, which may be, for instance, a liquid composition such as an aqueous liquid composition. For example, the at least one anionic polymer may range anywhere from 0.1% wt/wt to 0.2% wt/wt to 0.5% wt/wt to 1% wt/wt to 2% wt/wt to 5% wt/wt to 10% wt/wt to 20% wt/wt to 30% wt/wt to 40% wt/wt to 50% wt/wt with respect to the weight of the composition (e.g., ranging between any two of the preceding numerical values).

In some instances, the aqueous liquid embolic materials/composition may be disposed in a suitable delivery vessel such as a syringe. For example, the block copolymer may be disposed in the syringe. The templating agent may also be disposed in the syringe. Alternatively, the templating agent may be disposed in a separate delivery vessel (e.g., a second syringe). In such instances, the block copolymer and templating agent may be combined prior to delivery to the patient or the block copolymer and templating agent may be delivered to the patient separately (e.g., such that the block copolymer and the templating agent may combine in vivo). Kits are contemplated that include the block copolymer in the delivery vessel (e.g., a syringe). In such kits, the templating agent may also be disposed in the delivery vessel. Alternatively, the kit may include a second delivery vessel (e.g., a second syringe) containing the templating agent.

In some embodiments, the aqueous liquid embolic materials/compositions may further comprise at least one imaging agent and/or contrast agent. Some examples of imaging agents include radiocontrast agents, imageable radioisotopes, fluorescent dyes, magnetic resonance imaging (MRI) contrast agents, ultrasound contrast agents and near-infrared (NIR) imaging contrast agents. Particular examples of radiocontrast agents include metallic particles such as particles of tantalum, tungsten, rhenium, niobium, molybdenum, and their alloys, which metallic particles may be spherical or non-spherical. Particular examples of radiocontrast agents further include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®). Further particular examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd^{(III)}, Mn^{(II)}, Fe^{(III)} and compounds (including chelates) containing the same, such as gadolinium ion chelated with diethylenetriaminepentaacetic acid, (c) contrast agents for use in conjunction with ultrasound imaging, including organic and inorganic echogenic particles (e.g., particles that result in an increase in the reflected ultrasonic energy) or organic and inorganic echolucent particles (e.g., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and borondipyrromethane (BODIPY) analogs, among others, and (c) imageable radioisotopes including ^{99m}Tc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr and ¹⁷⁷Lu, among others.

In some instances, the at least one imaging agent is present in an amount ranging from 1% wt/wt or less to 70% wt/wt or more with respect to the weight of the composition, which may be, for instance, a liquid composition such as an aqueous liquid composition. For example, the at least one imaging agent may be present in the composition in a concentration ranging anywhere from 1% wt/wt to 2% wt/wt to 5% wt/wt to 10% wt/wt to 20% wt/wt to 30% wt/wt to 40% wt/wt to 70% wt/wt.

In some embodiments, the aqueous liquid embolic materials/compositions may further comprise one or more therapeutic agents. Some examples of therapeutic agents include small molecule therapeutic agents (defined herein as therapeutic agents having a molecular weight less than 2000 g/mol, typically less than 1500 g/mol, more typically less than 1000 g/mol), biomolecules (e.g., polypeptides including proteins and protein fragments, such as antibodies and antibody fragments and oligopeptides, as well as polynucleotides and oligonucleotides, including nucleic acids and nucleic acid analogs such as deoxyribonucleic acids, ribonucleic acids, peptide nucleic acids, and fragments thereof), and radioisotopes.

In some embodiments, the one or more therapeutic agent is present in an amount ranging from 0.001% wt/wt (10 ppm) or less to 80% wt/wt or more with respect to the weight of the composition, which may be, for instance, a liquid composition such as an aqueous liquid composition. For example, the one or more therapeutic agents may be present in the composition in a concentration ranging anywhere from 0.001% wt/wt to 0.002% wt/wt to 0.005% wt/wt to 0.01% wt/wt to 0.02% wt/wt to 0.05% wt/wt to 0.1% wt/wt to 0.2% wt/wt to 0.5% wt/wt to 1% wt/wt to 2% wt/wt to 5% wt/wt to 10% wt/wt to 20% wt/wt to 50% wt/wt to 80% wt/wt (e.g., ranging between any two of the preceding numerical values).

Some examples of therapeutic agents include anti-angiogenic agents, cytotoxic agents, chemotherapeutic agents, checkpoint inhibitors, immune modulatory cytokines, T-cell agonists, and STING (stimulator of interferon genes) agonists, among others.

Examples of therapeutic agents include: checkpoint inhibitors including inhibitors of the binding of PD-1 to PD-L1, inhibitors of the binding of CTLA-4 to CD80 and/or CD86, inhibitors of the binding of TIGIT to CD-112, and inhibitors of the binding of LAG-3 to MHC class II molecules; antibodies or antigen binding fragments thereof that bind to PD-1 (e.g., pembrolizumab, nivolumab domvanalimab, etc.), PD-L1 (e.g., atezolizumab, aveluma, durvalumab, etc.), LAG-3 (e.g., relatlimab, etc.), TIM-3 (e.g., LY3321367, MBG453, TSR-022, etc.), TIGIT (e.g., etigilimab, tiragolumab, vibostolimab, etc.), or CTLA-4 (e.g., ipilimumab tremelimumab, etc.); antibodies or antigen binding fragments thereof that bind to CD3, CD19, CD20, CD22, CD52, CD79B, CD30, CD33, CD38, CD52, CD79B, HER2, EGFR, VEGF, VEGFR2, EPCAM/CD3, GD2, IL-6, RANKL, SLAMF7, CCR4, PDGFRα, Nectin-4 or TROP2; immune modulatory cytokines such as IL-2, IL-12, IL-15, IL-23, interferon gamma (IFN-γ) and gm-CSF (granulocyte macrophage colony stimulating factor); T-cell agonists such as TLR3 agonists (e.g., polyinosinic: polycytidylic acid, double stranded RNAs, etc.), TLR7 agonists (e.g., TMX-202, gardiquimod, imiquimod, etc.), TLR8 agonists (e.g., VTX-2337, etc.), TLR7/8 agonists (e.g., MEDI9197, R848, resiquimod, etc.), TLR9 agonists (e.g., lefitolimod (MGN1703), tilsotolimod, CpG oligodeoxynucleotides (e.g., agatolimod), etc.); and STING agonists such as GSK 532, cyclic dinucleotides (e.g., cyclic guanosine monophosphate-adenosine monophosphate), CRD5500 (LB-061), E7766, ADU-S100, SB11285 MSA2, MK1454, TTI-10001, etc.), among others.

Examples of therapeutic agents also include: camptothecins (such as irinotecan, topotecan and exatecan) and anthracyclines (such as doxorubicin, daunorubicin, idarubicin and epirubicin), antiangiogenic agents (such as vascular endothelial growth factor receptor (VEGFR) inhibitors, such as axitinib, bortezomib, bosutinib canertinib, dovitinib, dasatinib, erlotinib gefitinib, imatinib, lapatinib, lestaurtinib, masutinib, mubitinib, pazopanib, pazopanib semaxanib, sorafenib, sunitinib, tandutinib, vandetanib, vatalanib and vismodegib), microtubule assembly inhibitors (such as vinblastine, vinorelbine and vincristine), aromatase inhibitors (such as anastrazole), platinum drugs (such as cisplatin, oxaliplatin, carboplatin and miriplatin), nucleoside analogues (such as 5-FU, cytarabine, fludarabine and gemcitabine), paclitaxel, docetaxel, mitomycin C, mitoxantrone, bleomycin, pingyangmycin, abiraterone, amifostine, buserelin, degarelix, folinic acid, goserelin, lanreotide, lenalidomide, letrozole, leuprorelin, octreotide, tamoxifen, triptorelin, bendamustine, chlorambucil, dacarbazine, melphalan, procarbazine, temozolomide, rapamycin (and analogues, such as zotarolimus, everolimus, umirolimus and sirolimus), antimetabolites such as 5-fluorouracil, multityrosine kinase inhibitors such as sorafenib, sunitinib, regorafenib, brivinb, dasetanib, bosutinib, erlotinib, gefitinib, imatinib and vandetinib, methotrexate, pemetrexed, raltitrexed, rivaroxaban, apixaban, combinations thereof, and/or the like.

Therapeutic radioisotopes include, but are not limited to, ¹⁷⁷Lu, ⁹⁰Y, ¹³¹I, ⁸⁹Sr, ¹⁵³Sm, ²²³Ra, ²²⁴Ra, ²¹¹At, ²²⁵Ac, ²²⁷Th, ²¹²Bi, ²¹³Bi, and/or ²¹²Pb.

In some embodiments, the compositions described herein may comprise therapeutic agents that are charged and/or uncharged at physiological pH. The charged therapeutic agents may be electrostatically held within the gel compositions and subsequently released therefrom by an ion exchange mechanism (e.g., where the compositions further comprise an anionic polymer that comprises one or more negatively charged groups selected from sulfonate groups, sulfate groups, phosphate groups, phosphonate groups or carboxylate groups). Charged therapeutic agents electrostatically held in the gel compositions may elute from the gel compositions in electrolytic media, such as physiological saline (0.90% w/v NaCl) or in-vivo, e.g., in the blood or tissues, to provide a sustained release of therapeutic agent over several hours, days or even weeks. Uncharged therapeutic agents in the gel compositions may also elute from the gel compositions in vivo. This may be particularly advantageous, for example, when rapid elution or a “burst effect” is desired, for example, for rapid therapeutic agent delivery to tissue, or when the low solubility of the therapeutic agent under physiological conditions determines the release profile rather than ionic interaction.

Embodiments of the present disclosure also relate to medical compositions that correspond to, or are formed from, the liquid compositions of any of the preceding embodiments. For example, as previous noted, such liquid compositions can be used for the in vivo formation of embolizations, fiducial markers, tissue bulking materials, tissue-spacing materials, and therapeutic agent depots.

Embodiments of the present disclosure further relate to medical procedures that employ the liquid compositions described herein. For example, in some embodiments, the medical procedures are methods of tissue embolization that comprise delivering the liquid compositions into one or more blood vessels feeding the tissue. Such procedures may be used to treat a variety of conditions including treatment of arteriovenous malformations, treatment of gastrointestinal bleeding, treatment of endoleaks, filling of aneurysms, treatment of a bleed caused by disease or trauma, treatment of solid tumors, particularly hyper-vascular tumors, such as those of the liver, prostate, kidney, brain, colon, bone and lung, as well as benign hyperplastic conditions such as treatment of prostate hyperplasia or treatment of uterine fibroids.

In some embodiments, the medical procedures are methods of local or systemic therapeutic agent release that comprise delivering (e.g., by injecting, spraying, etc.) the liquid compositions described herein to a patient (e.g., onto tissue of the patient, into tissue of the patient, between tissues of the patient, etc.).

In some embodiments, the medical procedures are methods of treatment that comprise delivering (e.g., by injecting, spraying, etc.) the liquid compositions described herein into or onto a tumor of a patient, wherein the therapeutic agent is released into the tumor.

In some embodiments, the medical procedures are methods of spacing a first tissue from a second tissue that comprise delivering (e.g., injecting, etc.) the liquid compositions described herein between the first tissue and the second tissue (e.g., between prostate tissue and rectal tissue).

In some embodiments, the medical procedures are methods of treatment that comprise delivering (e.g., by injecting, spraying, etc.) the liquid compositions described herein into a patient as a fiducial marker.

In yet further embodiments, the present disclosure relates to the use of the liquid compositions described herein in the manufacture of a medicament for the treatment of various diseases and conditions, including treatment of arteriovenous malformations, treatment of gastrointestinal bleeding, filling of aneurysms, treatment of solid tumors, particularly hypervascular tumors, such as those of the liver, prostate, kidney, brain, colon, bone and lung, as well as treatment of benign hyperplastic conditions such as prostate hyperplasia or uterine fibroids.

The present disclosure also relates to the use of any of the therapeutic agents described herein in the manufacture of a medicament for the treatment of such diseases and conditions wherein the therapeutic agents is incorporated into a liquid or gel compositions described herein. The present disclosure also relates to the use of any of the therapeutic agents herein in the treatment of such diseases and conditions wherein the therapeutic agents is incorporated into a liquid or gel compositions described herein. The liquid compositions may be particularly used where the liquid compositions are delivered by the transcatheter route, by injecting, by implanting, by spraying, etc.

U.S. patent application Ser. No. 17/992,662 is herein incorporated by reference.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An aqueous liquid embolic composition, comprising:
a di-block or tri-block copolymer including a charged block and a hydrophobic block; and
a templating agent.

2. The aqueous liquid embolic composition of claim 1, wherein the charged block includes polylysine.

3. The aqueous liquid embolic composition of claim 1, wherein the charged block includes polyornithine.

4. The aqueous liquid embolic composition of claim 1, wherein the charged block includes polyarginine.

5. The aqueous liquid embolic composition of claim 1, wherein the charged block includes polymethylarginine.

6. The aqueous liquid embolic composition of claim 1, wherein the charged block includes polyglutamic acid.

7. The aqueous liquid embolic composition of claim 1, wherein the charged block includes polyaspartic acid.

8. The aqueous liquid embolic composition of claim 1, wherein the charged block includes a natural or non-natural amino acid with a pKa from 3-11.

9. The aqueous liquid embolic composition of claim 1, wherein the hydrophobic block includes a hydrophobic amino acid.

10. The aqueous liquid embolic composition of claim 1, wherein the hydrophobic block includes polyphenylalanine.

11. The aqueous liquid embolic composition of claim 1, wherein the hydrophobic block includes one or more of polyleucine, polyisoleucine, polytryptophan, and polytyrosine.

12. The aqueous liquid embolic composition of claim 1, wherein the hydrophobic block includes one or more natural or non-natural amino acids that have pendant aliphatic, cycloaliphatic, or aromatic side chains.

13. The aqueous liquid embolic composition of claim 1, wherein the composition becomes a gel when injected into a vasculature of a patient.

14. The aqueous liquid embolic composition of claim 1, wherein the composition includes a di-block copolymer.

15. The aqueous liquid embolic composition of claim 1, wherein the templating agent includes one or more of gelatin, hyaluronic acid, carboxy methylcellulose, poly(2-acrylamido-2-methyl-1-propanesulfonic acid), chitosan, alginate, chondroitan sulfate, polydiallyldimethylammonium chloride, polylysine, polyarginine, polyallylamine, polysodium acrylate, sulfonated polystyrene, polyvinyl sulfonic acid, poly N,N-dimethyl-3,5-dimethylene piperidinium chloride, poly(vinylbenzyl trimethylammonium) chloride, polyvinyl amine, carrageenan, pectin, xanthan gum, and guar gum.

16. An aqueous liquid embolic composition, comprising:
a block copolymer including a charged block and a hydrophobic block;
wherein the charged block includes a charged amino acid; and
wherein the hydrophobic block includes a hydrophobic amino acid.

17. The aqueous liquid embolic composition of claim 16, wherein the block copolymer includes a di-block copolymer.

18. A kit for embolization, the kit comprising:
a syringe containing a block copolymer including a charged block and a hydrophobic block;
wherein the charged block includes a charged (cationic or anionic) amino acid;
wherein the hydrophobic block includes a hydrophobic amino acid; and
a templating agent.

19. The kit of claim 18, wherein the templating agent is disposed within the syringe.

20. The kit of claim 19, further comprising a second syringe containing the templating agent.
